(19) United States
(12) Patent Application Publication
(10) Pub. No.: US 2003/0018088 A1
(43) Pub. Date: Jan. 23, 2003

(54) Fischer-tropsch process using sponge cobalt catalyst

(75) Inventors: Ajoy P. Raje, Stillwater, OK (US); Theodore A. Koch, Wilmington, DE (US); Sourav Kumar Sengupta, Wilmington, DE (US); Mark Jay Harper, (US); Gloria I. Straguzzi, Ponca City, OK (US); Hasan Dindi, Wilmington, DE (US)

Correspondence Address: Joanna K. Payne, CONOCO INC., 1000 South Pine, 2635W, P.O. Box 1267, Ponca City, OK, 74602-1267, US

(73) Assignee: CONOCO INC., Houston, TX

(21) Appl. No.: 10/085,932

(22) Filed: Feb. 28, 2002

Related U.S. Application Data
(60) Provisional application No. 60/272,281, filed on Feb. 28, 2001.
(60) Provisional application No. 60/287,356, filed on Apr. 30, 2001.

(51) Int. Cl. C07C 27/06

(52) U.S. Cl. 518/715

(57) ABSTRACT

A process is disclosed for the hydrogenation of carbon monoxide. The process involves contacting a feed stream comprising hydrogen and carbon monoxide with a catalyst in a reaction zone maintained at conversion-promoting conditions effective to produce an effluent stream, preferably comprising hydrocarbons. The catalyst used in the process is in the form of a sponge. The process is preferably adapted to produce hydrocarbons suitable for the production of diesel fuel. The catalyst used in the process includes at least one catalytic metal for Fischer-Tropsch reactions, preferably cobalt. Preferably the catalyst further includes at least one promoter suitable for the Fischer-Tropsch reaction, such as at least one element selected from among Groups 2-15 of the Periodic Table, preferably at lease one of chromium, iron, molybdenum, nickel, palladium, platinum, rhenium, rhodium, ruthenium, and combinations thereof. Preferably the catalyst further includes at least one of aluminum, silicon, titanium, and zirconium, and combinations thereof.

### CROSS-REFERENCE TO RELATED APPLICATIONS

This application claims the benefit under 35 U.S.C. §119(e) of U.S. Provisional Patent Application No. 60/272,281, filed Feb. 28, 2001, and U.S. Provisional Patent Application No. 60/287,356, filed Apr. 30, 2001, each of which is hereby incorporated herein by reference.

### STATEMENT REGARDING FEDERALLY SPONSORED RESEARCH OR DEVELOPMENT

Not applicable.

### FIELD OF THE INVENTION

The present invention relates to a process for the hydrogenation of carbon monoxide to produce hydrocarbons. More particularly, the present invention relates to a process for the hydrogenation of carbon monoxide in the presence of a cobalt sponge catalyst to selectively produce hydrocarbons suitable for the production of diesel fuel.

### BACKGROUND OF THE INVENTION

Liquid hydrocarbons serve a number of important purposes and are an invaluable source of gasoline and diesel fuel. Historically, such hydrocarbons have been obtained through drilling and extraction from oil reserves. Unfortunately, though, these reserves represent an exhaustible supply that is quickly being depleted. Alternatively, liquid hydrocarbons can be synthesized from natural gas, a mixture of short-chain hydrocarbons including principally methane. As the oil reserves are depleted, this approach is becoming an increasingly attractive method of acquiring longer chain hydrocarbons, in part because the natural gas reserve is expected to significantly outlast the remaining oil reserves.

The conversion of methane to hydrocarbons is typically carried out in two steps. In the first step, methane is converted into a mixture of carbon monoxide and hydrogen, commonly referred to as synthesis gas or syngas. In a second step, the synthesis gas is converted into various hydrocarbons. This second step, the preparation of hydrocarbons from synthesis gas, is well known in the art and is usually referred to as a Fischer-Tropsch synthesis, Fischer-Tropsch process, or Fischer-Tropsch reaction. Fischer-Tropsch synthesis generally entails contacting a stream of synthesis gas with an appropriate catalyst under temperature and pressure conditions that favor the formation of hydrocarbon products. The product stream prepared by using these catalysts usually includes a mixture of hydrocarbons having a very wide range of molecular weights. Product distribution or product selectivity depends heavily on the type and structure of the catalysts and on the reactor type and operating conditions. Accordingly, in synthesizing diesel fuels it is highly desirable to maximize the selectivity and yield of the Fischer-Tropsch synthesis to the production of high-value liquid hydrocarbons.

Catalysts for use in the Fischer-Tropsch synthesis usually contain a catalytic metal of Groups 8, 9, or 10 (in the new notation of the periodic table of the elements, which is followed throughout). In particular, iron, cobalt, nickel, and ruthenium have commonly been used as the catalytically active metals. Nickel catalysts favor termination and are useful for aiding the selective production of methane from syngas. Iron has the advantage of being readily available and relatively inexpensive but the disadvantage of a water-gas shift activity. Ruthenium has the advantage of high activity but unfortunately is quite expensive. Consequently, although ruthenium is not the economically preferred catalyst for commercial Fischer-Tropsch production, it is often used in low concentrations as a promoter with one of the other catalytic metals. Cobalt has the advantages of being more active than iron and more economically feasible than ruthenium. Further, cobalt is less selective to methane than nickel.

Accordingly, cobalt has been extensively investigated as a catalyst for the production of hydrocarbons with weights corresponding to the range of the gasoline, diesel, and higher weight fractions of crude oil. In particular, cobalt has been found to be suitable for catalyzing a process in which synthesis gas is converted to hydrocarbons having primarily five or more carbon atoms (i.e., where the C₅₊ selectivity of the catalyst is high). See, for example, H. Schulz, Short History and Present Trends of Fischer-Tropsch Synthesis, APPLIED CATALYSIS A, vol. 186, pp. 3-12 (1999), which is hereby incorporated herein by reference in its entirety.

Catalyst systems often employ a promoter in conjunction with the principal catalytic metal. A promoter typically improves a measure of the performance of a catalyst, such as productivity, lifetime, selectivity, or regenerability. Well-known Fischer-Tropsch promoters include rhenium, ruthenium, platinum, and metal oxides. Metal oxide promoters tend to be difficult to reduce and thus remain as the oxide in an activated catalyst.

Catalysts conventionally include a support material. The support material serves as a carrier for the catalytic metal and any promoter deposited on the support and is typically porous. Catalyst supports for catalysts used in Fischer-Tropsch synthesis of hydrocarbons have typically been refractory oxides (e.g., silica, alumina, titania, thoria, zirconia or mixtures thereof, such as silica-alumina). A disadvantage of some support materials, such as alumina, is the tendency for metal support interactions to occur that tend to impede the reducibility of cobalt in cobalt-based catalysts. Typically a supported catalyst requires activation by reduction in hydrogen to convert catalytic metal present in compounds (e.g. oxides) thereof to the metallic (completely reduced) state.

Thus, it has been customary to add to a cobalt-based catalyst one of the precious metal promoters, such as ruthenium, rhenium, and platinum, that are known to increase the reducibility of cobalt. However, ruthenium, rhenium, and platinum are each rare and costly. Thus, although these promoters are used at relatively low concentrations, they contribute significantly to the cost of Fischer-Tropsch catalysis.

Research continues on the development of more efficient Fischer-Tropsch catalyst systems and reaction systems that increase the selectivity for high-value hydrocarbons in the Fischer-Tropsch product stream. The products of the Fischer-Tropsch hydrogenation reaction can range from molecules containing a single carbon to those containing ten, fifteen or more carbons. In any Fischer-Tropsch synthesis process, the range of molecular weights in the direct product of the synthesis depends on the catalytic mechanism of formation of carbon-carbon bonds that increase the length of a hydrocarbon. Typically, in the Fischer-Tropsch synthesis, the distribution of weights that is observed such as for C₅₊ hydrocarbons, can be described by likening the Fischer-Tropsch reaction to a polymerization reaction with an Anderson-Shultz-Flory chain growth probability (α) that is independent of the number of carbon atoms in the lengthening molecule. (Throughout the specification Cₙ₊ denotes hydrocarbons containing at least n hydrocarbons, that is n or more hydrocarbons). Thus, a range of hydrocarbons from C₁ to C₂₁₊ may be formed, with a selectivity to liquids that depends on the production of gaseous hydrocarbons, as well as on α. In particular, the selectivity to liquids in the non-gaseous product typically is characterized by α. α is typically interpreted as the ratio of the concentration of Cₙ₊ product to the concentration of Cₙ product. A value of α of at least 0.72 is preferred for producing high carbon-length hydrocarbons, such as those of diesel fractions.

There are continuing efforts to find catalysts and processes that are more effective at yielding high-value products. The high-value products include gasoline, diesel fuel, jet fuel, and various other relatively valuable hydrocarbons that are, notably, liquids at room temperature. It is highly desirable to maximize the production of high-value liquid hydrocarbons, such as hydrocarbons with 5 to 20 carbon atoms per hydrocarbon chain (C₅-C₂₀ hydrocarbons). These include, for example, wide range naphtha fractions, such as fractions containing C₅-C₁₂ hydrocarbons, useful for processing to gasoline, and gasoil fractions, such as fractions containing C₁₃-C₂₀ hydrocarbons, useful for processing to diesel oil. The range of hydrocarbon chain lengths in naphtha and gasoil fractions varies in the art, and may depend on whether kerosene is included in naphtha, gasoil, or neither. Further, a division between a naphtha and a gasoil fraction may depend on the particular boiling points used to separate the fractions by distillation, and on the degree of branching of the hydrocarbons, since degree of branching of a hydrocarbon affects its boiling point.

Products with both lower and higher molecular weights than those typical of liquid products are less desirable in a Fischer-Tropsch process optimized for liquid production. Lower molecular weight products, such as C₁-C₄ hydrocarbons, tend to be gaseous at room temperature. The lightest of these is methane, which is the original gas that is converted into synthesis gas in the first step of the two-step process of converting methane to hydrocarbons. For this reason, and because methane is a gas at room temperature, methane is not typically one of the desired products and its formation is generally regarded as undesirable. Further, higher molecular weight products, such as C₂₁₊ hydrocarbons tend to be solid at room temperature, forming wax. A wax fraction typically includes C₂₁₊ hydrocarbons, but it may vary in its minimum hydrocarbon chain length depending on process conditions, similarly to naphtha and gasoil. Hydrocarbon waxes are conventionally subjected to an additional processing step such as hydrocracking for conversion to liquid hydrocarbons, typically when the hydrocarbon waxes are present at greater than 5 wt. % in the Fischer-Tropsch product.

Research continues on the development of more efficient but lower cost Fischer-Tropsch catalyst systems and reaction systems that also have other advantageous properties of durability, such as resistance to localized heating and attrition resistance. The Fischer-Tropsch synthesis is exothermic, that is, it gives off heat. The catalyst, particularly a supported catalyst, may develop hot spots as a result of localized heating. Therefore it is desirable to develop catalysts that are less susceptible to localized heating. Further, it is desirable to develop catalysts that are attrition resistant.

Despite the vast amount of research effort in this field, Fischer-Tropsch catalysts that can be used to more economically produce liquid hydrocarbon products, particularly diesel oil fractions, are desired. There is still a great need to identify effective Fischer-Tropsch processes using catalysts for Fischer-Tropsch synthesis in which the catalyst is selective to C₅-C₂₀ hydrocarbons, so as to maximize the value of the hydrocarbons produced and thus maximize the process economics. For successful operation on a commercial scale, the Fischer-Tropsch process must be able to achieve a high conversion of the methane feedstock at high gas hourly space velocities, while maintaining high selectivity of the process to the desired products. Accordingly, it is desired to provide processes in which more durable and economical catalysts are active for hydrogenation of carbon monoxide and selective towards liquid hydrocarbons.

### SUMMARY OF THE INVENTION

This invention relates to a process for producing hydrocarbons, and includes using a cobalt-containing catalyst in the form of a sponge. The present Fischer-Tropsch synthesis process includes contacting a feed stream comprising hydrogen and carbon monoxide with this catalyst in a reaction zone maintained at conversion-promoting conditions effective to produce an effluent stream including hydrocarbons.

The present Fischer-Tropsch synthesis is preferably adapted to produce hydrocarbons suitable for the production of diesel fuel. The C₅₊ hydrocarbons preferably have a distribution of molecular weights described by an α of at least about 0.72. The selectivity to methane is preferably not more than about 20%. In some embodiments, the selectivity to wax is not more than about 5%. Alternatively, in other embodiments, the selectivity to wax is greater than about 5% In some embodiments, the reaction zone includes at least one slurry bubble column. Alternatively, in some other embodiments, the reaction zone includes at least one fixed bed reactor.

The catalyst preferably contains a catalytically active metal including from about 85 to about 99 wt. % cobalt. The catalyst preferably further includes from about 0.05 to about 6 wt % of a second metal. The second metal is preferably at least one element selected from the group consisting of Groups 2-15 of the Periodic Table, more preferably selected from the group consisting of chromium, iron, molybdenum, nickel, palladium, platinum, rhenium, rhodium, ruthenium, and combinations thereof. The second metal is preferably a promoter for the Fischer-Tropsch reaction. Still further, the present catalyst preferably includes from about 1 to about 15 wt % of at least one structural material selected from the group consisting of aluminum, silicon, titanium, zirconium, and combinations thereof.

The present Fischer-Tropsch synthesis may include regenerating the catalyst, and cycling between hydrocarbon production and catalyst regeneration. Regeneration preferably includes stripping the catalyst with hydrogen at reaction conditions and reducing the catalyst with hydrogen at a temperature elevated above the reaction temperature and at reaction pressure. In some embodiments, the regeneration is carried out in situ. Alternatively, in other embodiments, the regeneration is carried out ex situ.

### DESCRIPTION OF THE DRAWINGS

For an introduction to the detailed description of the preferred embodiments of the invention, reference will now be made to the accompanying drawings, wherein:

FIG. 1 is a plot of the performance of a sponge cobalt-containing catalyst in the Fischer-Tropsch reaction a slurry phase reactor.

FIG. 2 is a plot indicating the performance of a sponge cobalt-containing catalyst in the Fischer-Tropsch reaction in a slurry phase reactor before and after a regeneration procedure. Each rise of activity, indicated by an arrow, occurs after regeneration.

### DETAILED DESCRIPTION OF A PREFERRED EMBODIMENT

Catalyst

Catalysts that are contemplated by the present method include those in the form of a sponge of a Fischer-Tropsch catalytic metal, such as cobalt, cobalt/ruthenium, cobalt/ruthenium/molybdenum, and cobalt/chromium/nickel. The amount of cobalt present in the catalyst may vary. A particularly preferred catalyst includes cobalt in an amount totaling from about 85 to about 99% by weight (as the metal) of the total weight of catalyst, preferably from about 92 to 97% by weight. The catalyst preferably further includes ruthenium. In a preferred embodiment, ruthenium is added to the cobalt catalyst in a concentration sufficient to provide a weight ratio of elemental ruthenium to elemental cobalt of from about 0.05 to about 7% by weight (dry basis), preferably from about 0.05 to about 0.3% by weight, still more preferably from about 0.1 to about 0.3% by weight. The catalyst preferably further includes aluminum in an amount totaling from about 1 to about 15% by weight (as the metal) of the total weight of the catalyst, and preferably from about 1 to about 10% by weight, still more preferably from about 2 to about 8% by weight. Aluminum is preferably present as a stabilizing material in an amount sufficient to help provide stability to the catalyst structure and in an amount low enough so as not to significantly reduce available surface area of catalytic metal. Alternately, the catalyst may include silicon, zirconium, or titanium as a stabilizing material, preferably from about 1 to about 15% by weight, more preferably from about 1 to about 10% by weight, more preferably from about 2 to about 8% by weight.

The catalyst is preferably in the form of finely-divided particles of sponge metal. The metal is preferably a Fischer-Tropsch metal, more preferably cobalt. Cobalt has the advantages of being more active than iron, less selective to CO₂ than iron, more available than ruthenium, and less selective to methane than nickel. A sponge metal has an extended porous skeletal structure of metal, similar in form to natural sponge. The sponge metal may include dissolved aluminum, silicon, titanium, or zirconium. The sponge cobalt may optionally include at least one promoter. The promoter may be selected from among the elements of Groups 2-15 of the Periodic Table of the Elements. Sponge cobalt containing a promoter selected from the group consisting of nickel, palladium, platinum, rhenium, rhodium, chromium, iron, molybdenum, ruthenium, and combinations thereof are particularly preferred. In a preferred embodiment, the promoter is added to the catalyst in an amount totaling from about 0.05 to about 6% by weight (as the metal) of the total weight of catalyst, more preferably from about 0.05 to about 0.3% by weight, still more preferably from about 0.05 to about 0.3% by weight. The sponge metal catalyst preferably contains surface hydrous oxides, adsorbed hydrogen radicals, and hydrogen bubbles in the pores. Sponge metal catalysts of varying compositions, including sponge cobalt, are commercially available from W.R. Grace & Co. and from Activated Metals. A process for preparing sponge metal catalyst is disclosed in EPO Application No. 0,212,986. Preparation of shaped Raney™ catalyst is disclosed in U.S. Pat. Nos. 4,826,799 and 4,895,994. The Raney™ process is known in the art and conventionally includes providing an alloy of a catalytic metal with aluminum. The alloy is crushed to a fine powder and aluminum is removed by leaching with a solution of a strong base, such as sodium hydroxide, leaving a finely divided catalytic sponge metal. A preferred procedure for treating an alloy with sodium hydroxide to remove aluminum from the alloy is described, for example, in U.S. Pat. No. 6,156,694. The incorporation of a promoter by post-treatment of a sponge catalyst, using a salt of the promoter is disclosed in British Patent GB 1,119,512 and French Patent FR 2,722,710. A preferred procedure for incorporation of a promoter by post-treatment of a sponge catalyst is described in PCT Publication WO 00/67903. Each of the references listed in the present paragraph is hereby incorporated herein by reference.

Catalysis

The feed gas charged to the synthesis process includes hydrogen, or a hydrogen source, and carbon monoxide. H₂/CO mixtures suitable as a feedstock for conversion to hydrocarbons according to the synthesis process can be obtained from light hydrocarbons such as methane by means of steam reforming, partial oxidation, or other processes known in the art. Preferably the hydrogen is provided by free hydrogen, although some Fischer-Tropsch catalysts have sufficient water gas shift activity to convert some water to hydrogen for use in the Fischer-Tropsch process. It is preferred that the molar ratio of hydrogen to carbon monoxide in the feed be greater than 0.5:1 (e.g., from about 0.67:1 to 2.5:1), more preferably at least 1.5:1. The feed gas stream may contain hydrogen and carbon monoxide in a molar ratio of about 2:1. The feed gas stream may also contain carbon dioxide. The feed gas stream should contain a low concentration of compounds or elements that have a deleterious effect on the catalyst, such as poisons. For example, the feed gas may need to be pre-treated to ensure that it contains low concentrations of sulfur or nitrogen compounds such as hydrogen sulfide, ammonia and carbonyl sulfides.

The feed gas is contacted with the catalyst in a reaction zone. Mechanical arrangements of conventional design may be employed as the reaction zone including, for example, plugged flow, continuous stirred tank, fixed bed, fluidized bed, slurry phase, slurry bubble column, reactive distillation column, or ebulliating bed reactors, among others, may be used. A slurry bubble column reactor is described in U.S. Pat. No. 4,429,159, hereby incorporated herein by reference. Plug flow, fluidized bed, reactive distillation, ebulliating bed, and continuous stirred tank reactors have been delineated in “Chemical Reaction Engineering,” by Octave Levenspiel, and are known in the art. The size and physical form of the catalyst may vary, depending on the reactor in which it is to be used.

When the reaction zone includes a slurry bubble column, the column preferably includes a three-phase slurry. Further, a process for producing hydrocarbons by contacting a feed stream including carbon monoxide and hydrogen with a catalyst in a slurry bubble column, preferably includes dispersing the particles of the catalyst in a liquid phase comprising the hydrocarbons so as to form a two-phase slurry; and dispersing the hydrogen and carbon monoxide in the two-phase slurry so as the form the three-phase slurry. Further, the slurry bubble column preferably includes a vertical reactor and dispersal preferably includes injection and distribution in the bottom half of the reactor. Alternatively, dispersal may occur in any suitable alternative manner, such as by injection and distribution in the top half of the reactor.

The Fischer-Tropsch process is typically run in a continuous mode. In this mode, the gas hourly space velocity through the reaction zone may range from about 0.5 Normal liters of syngas/hr/gram catalyst to about 15 Normal liters of syngas/hr/gram catalyst, preferably from about 1 Normal liters of syngas/hr/gram catalyst to about 10 Normal liters of syngas/hr/gram catalyst. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psig (653 kPa) to about 1000 psig (6994 kPa), preferably from 160 psig (653 kPa) to about 600 psig (4237 kPa).

The products resulting from Fischer-Tropsch synthesis will have a range of molecular weights. Typically, the carbon number range of the product hydrocarbons will start at methane and continue to the limits observable by modem analysis, about 50 to 100 carbons per molecule. The catalyst of the present process is particularly useful for making hydrocarbons having five or more carbon atoms, especially when the above-referenced preferred space velocity, temperature and pressure ranges are employed. In particular, the product hydrocarbons are preferably described by an α of at least about 0.72. Further, the methane selectivity is preferably not more than about 20% by weight and the wax selectivity is preferably not more than about 5% by weight.

The effluent stream of the reaction zone may be cooled to effect the condensation of hydrocarbons, for example those liquid under standard conditions of ambient temperature and pressure and passed into a separation zone separating the vapor phase products from effluent stream. The vapor phase material may be passed into a second stage of cooling for recovery of additional hydrocarbons. The remaining effluent stream together with any liquid from a subsequent separation zone may be fed into a fractionation column. Typically, a stripping column is employed first to remove light hydrocarbons such as propane and butane. Further, typically the effluent stream is treated to remove any alcohols and hydrogenate any olefins. The remaining hydrocarbons may be passed into a fractionation column where they are separated by boiling point range into products such as naphtha, kerosene and fuel oils. Hydrocarbons recovered from the reaction zone and having a boiling point above that of the desired products may be passed into conventional processing equipment such as a hydrocracking zone in order to reduce their molecular weight. The gas phase recovered from the reactor zone effluent stream after hydrocarbon recovery may be partially recycled if it contains a sufficient quantity of hydrogen and/or carbon monoxide. Further, lighter hydrocarbon products, such as gasoline weight ranges hydrocarbons, for example C₅-C₁₂ hydrocarbons, may be recycled to increase the yield of diesel weight range hydrocarbons.

Regeneration

According to an embodiment of the present invention, a sponge cobalt-containing catalyst is regenerated by the following procedure that includes at least one of the steps of (a) contacting the catalyst with hydrogen at a temperature and a pressure about equal to the Fischer-Tropsch reaction temperature and pressure, respectively; and (b) contacting the catalyst with hydrogen at a pressure substantially equal to the Fischer-Tropsch reaction pressure and a temperature elevated above the Fischer-Tropsch reaction temperature, for example by about 60° C. Step (a) is termed stripping herein, whereas step (b) is termed reduction herein. Regenerating the catalyst preferably includes both steps (a) and (b), which may be carried out in any order. Further, step (a) preferably precedes step (b).

The process of regenerating the catalyst is preferably performed in situ. That is, regenerating the catalyst occurs in the same vessel, reactor, apparatus, or reaction zone as the contacting of the catalyst with a feed gas in the Fischer-Tropsch synthesis. Further, a process for producing hydrocarbons may include contacting a feed stream comprising carbon monoxide and hydrogen with a sponge catalyst comprising cobalt in a reaction zone under reaction conditions effective to produce an effluent stream comprising C₅-C₂₀ hydrocarbons, regenerating the catalyst in situ, and cycling between reaction and regeneration. The regeneration may include stripping the catalyst with hydrogen at reaction conditions, and reducing the catalyst with hydrogen at an elevated temperature and at reaction pressure.

Further, it will be understood that although the above-described regeneration procedure is preferably performed in situ, contemplated embodiments includes performance of any of the above-described procedures ex situ. Ex situ regeneration may be performed for example in a regeneration zone. The regeneration zone may include a separate vessel from any reaction vessel contained in the reaction zone.

Without further elaboration, it is believed that one skilled in the art can, using the description herein, utilize the present invention to its fullest extent. The following embodiments are to be construed as illustrative, and not as constraining the scope of the present invention in any way whatsoever. For example, while the C₅-C₁₂, C₁₃-C₂₀, and C₂₁₊ fractions are illustrative of gasoline, diesel, and wax fractions of the hydrocarbon products, respectively, it will be understood that because the fractions are typically defined according to distillation temperature, the range of hydrocarbon lengths in each fraction of a Fischer-Tropsch product may vary, for example due to variable degree of branching of the hydrocarbons.

### EXAMPLES

General Procedure for Fixed Bed Testing

A. Automated Six-Reactor Catalyst Testing Unit

The catalyst testing unit was composed of a syngas feed system, a set of six tubular reactors, each reactor containing a set of wax and cold traps, back pressure regulators for each reactor, and three gas chromatographs (one on-line and two off-line). The entire system was controlled by a Programmable Logic Control system with a PC-based Operator Interface.

The syngas supply system included a hydrogen manifold, a carbon monoxide manifold and a nitrogen manifold to supply each of these gases to each of the reactors. Each manifold involved multiple pressurized gas cylinders, individual mass flow controllers and back pressure regulators. Before being fed to the reactors the carbon monoxide was purified over a 22% lead oxide on alumina catalyst placed in a trap to remove any iron carbonyls present. The individual gases or mixtures of these gases were mixed in a 300 cc vessel filled with glass beads before entering the common supply manifold feeding the six reactors.

The reactors were made of ⅜ in. O.D., ¼ in. I.D. stainless steel tubing. The length of the reactor tubing was 14 inches The actual length of the catalyst bed was 10 inches with 2 inches of 25/30 mesh (600-710 micron) glass beads and glass wool at the inlet and outlet of the reactor. The temperature of each reactor was measured by a four-point 1/16 in. multicouple placed axially inside the reactor. Each reactor was covered by a 1 in. O.D. copper sleeve for temperature uniformity and placed in a vertically-mounted tube furnace. The temperature of each reactor was controlled by a thermocouple placed in the copper sleeve.

The wax and cold traps were made of 75 cc pressure cylinders. The wax traps were set at 140° C. while the cold traps were set at 0° C. Each reactor had two wax traps in parallel followed by two cold traps in parallel. At any given time products from the reactor flowed through one wax and one cold trap in series. Following a material balance period, the hot and cold traps were switched to the other set in parallel. The wax traps collected a heavy hydrocarbon product distribution, usually C₆₊, while the cold traps collected a lighter hydrocarbon product distribution usually between C₃ and C₂₀. Water, a major byproduct of the F-T synthesis, was collected in both traps.

The back pressure regulator for each reactor was placed downstream of the wax and cold traps. It relieved the pressure from reaction pressure to ambient. Flow meters placed downstream of the back pressure regulators measured the flow rate of uncondensed gas products from each reactor.

All six reactors shared an on-line gas chromatograph. An eight port valve placed downstream of the flow meters determined which reactor product gases were fed at any given time to the on-line gas chromatograph.

B. Analytical Equipment/Procedures

The uncondensed gaseous products from the reactors were analyzed using a common on-line HP Refinery Gas Analyzer. This analyzer included 5 columns and four switching valves. The columns used were: (i) 2 ft×⅛ in SS 20% Sebaconitrile on 80/100 mesh Chromasorb (ii) 30 ft×⅛ in SS 20% Sebaconitrile on 80/100 mesh Chromasorb (iii) 6 ft×⅛ in SS Porapak Q 80/100 mesh (iv) 10 ft×⅛ in SS Molecular Sieve 13×45/60 mesh (v) 4 ft×⅛ in SS Molecular Sieve 13×45/60 mesh.

The Refinery Gas Analyzer was equipped with two thermal conductivity detectors and measured the concentrations of CO, H₂, N₂, CO₂, CH_{4,} and C₂ to C₅ alkenes/alkanes/isomers in the uncondensed reactor products.

The products from each of the wax and cold traps were separated into an aqueous and an organic phase. The organic phase from the hot trap was usually solid at room temperature. A portion of this solid product was dissolved in carbon disulfide before analysis. The organic phase from the cold trap was usually liquid at room temperature and was analyzed as obtained. The aqueous phases from the two traps were combined and analyzed for alcohols and other oxygenates.

Two off-line gas chromatographs equipped with flame ionization detectors were used for the analysis of the organic and aqueous phases collected from the wax and cold traps. The GC column used for the organic phase was a 60 m×250 micron HP-1 (crosslinked methyl siloxane) from Hewlett Packard. This column separates the organic phase into individual hydrocarbon compounds in the range C₃ to C₄₀. Hydrocarbons containing more than 40 carbon atoms were below the limit of detection for this chromatograph. However the inability to account for C₄₀₊hydrocarbons did not have an effect on the calculation of the a value. The GC column used for the aqueous phase was a 15 m×320 micron HP-5MS (crosslinked 5% phenyl methyl siloxane) from Hewlett Packard.

C. Catalyst Loading

Three grams of catalyst to be tested were mixed with 4 grams of 25/30 mesh (600-710 microns) and 4 grams of 2 mm glass beads. The 14-in. tubular reactor was first loaded with 25/30 mesh (600-710 microns) glass beads so as to occupy a 2-inch length of the reactor. The catalyst/glass bead mixture was then loaded, occupying 10 inches of the reactor length. The remaining 2 inches of reactor length was once again filled with 25/30 mesh (600-710 microns) glass beads. Both ends of the reactor were plugged with glass wool. A copper sleeve was placed around the reactor, which was then placed in the furnace. The sponge catalysts were stored under water before loading in the reactor. A water suspension of the sponge catalysts was loaded in the reactor to prevent exposure to air.

D. Start-up of Reaction

Sponge catalysts were not activated prior to reaction. A drying step usually preceded reaction to remove the water used during catalyst loading. During the drying step, the reactor was kept under nitrogen flow (100 cc/min & 40 psig) at room temperature for 2 hours. The reactor was then heated to 60° C. under nitrogen flow (100 cc/min & 40 psig) at a rate of 1° C./min. The reactor was maintained at this temperature for 2 hours. Following this step, the reactor was heated to 120° C. under nitrogen flow (100 cc/min & 40 psig) at a rate of 1.5° C./min and maintained at this temperature for 14 hours. The reactor was then pressurized to the desired reaction pressure (usually 350 psig). Syngas flow, with a 2:1 H₂/CO ratio, was fed to the reactor and the temperature was increased to the desired reaction temperature (usually 220° C.) at a rate of 1.5° C./min.

General Procedure for Slurry Bed Testing

A. Slurry Continuous-Flow Stirred Tank Reactor Unit (CSTR)

The slurry CSTR catalyst testing unit was composed of a gas feed system, a slurry stirred tank reactor, wax and cold traps, back pressure regulator, and three gas chromatographs (one on-line and two off-line).

The gas supply system involved multiple pressurized gas cylinders, pressure regulators and individual mass flow controllers to supply carbon monoxide, hydrogen and/or nitrogen to the reactor. The carbon monoxide was purified before being fed to the reactors over a 22% lead oxide on alumina catalyst placed in a trap to remove any iron carbonyls present.

The reactor was a 1 liter stainless-steel stirred autoclave equipped with two stirrers on a single shaft. The bottom stirrer was a gas-entrainment impeller while the top stirrer was a pitched turbine impeller. A thermocouple inside a well in the reactor measured the slurry temperature in the reactor. The reactor had a furnace for heating. The temperature of the reactor was controlled by a thermocouple measuring the furnace temperature. Gas feed to the reactor was at the bottom of the reactor, just below the bottom stirrer, through a ⅛ in. tube. Unconverted reactants and reactor products exited the reactor at the top through an in-line sintered metal filter.

The wax and cold traps used were made of 500 cc pressure cylinders. The wax traps were set at 100° C. while the cold traps were set at 0° C. The wax traps collected a heavy hydrocarbon product distribution usually C₆₊ while the cold traps collected a lighter hydrocarbon product distribution usually between C₃ and C₂₀. Water, a major byproduct of the Fischer-Tropsch is collected in both the traps.

The back pressure regulator for each reactor was placed downstream of the wax and cold traps. It relieved the pressure from reaction pressure to ambient. An electronic soap bubble flow meter, placed downstream of the back pressure regulator, was used to periodically measure the flow rate of the uncondensed gas products.

B. Analytical Equipment/Procedures

The uncondensed gaseous products from the reactors were analyzed using a HP MicroGC gas chromatograph. The chromatograph included four measurement channels and four thermal conductivity detectors. The chromatograph measured the concentrations of CO, H₂, N₂, CO₂, CH₄, C₂ to C₉ alkenes/alkanes/isomers in the uncondensed reactor products.

The products from the hot and cold traps were separated into an aqueous and an organic phase. The organic phase from the hot trap was usually solid at room temperature. A portion of this solid product was dissolved in carbon disulfide before analysis. The organic phase from the cold trap was usually liquid at room temperature and was analyzed as obtained. The aqueous phase from the two traps was combined and analyzed for alcohols and other oxygenates.

Two off-line gas chromatographs were used for the analysis of the organic and aqueous phases collected from the wax and cold traps. The chromatograph for the organic phase had a flame ionization detector and a DB-1 column for separation. This column separated the organic phase into individual hydrocarbon compounds in the range C₃ to C₄₅. Hydrocarbons containing more than 45 carbon atoms were below the limit of detection for this chromatograph. The chromatograph for the aqueous phase had a thermal conductivity detector and a packed Porpak-Q column for separation.

C. Catalyst Loading Catalyst Activation & Start-up of Reaction

1. Sponge Catalysts

The sponge catalysts were stored under water before loading in the reactor. 300 grams of a heavy hydrocarbon wax with an average molecular weight of 1400 was loaded in the reactor. The reactor was heated to 120° C. to melt the solid start-up wax. A water suspension of a known weight of the Raney-type catalysts was loaded in the reactor to prevent exposure to air. The reactor was then closed and the stirrer started at 1000 rpm to keep the catalyst suspended.

Sponge catalysts were not activated prior to reaction. A drying step usually preceded the reaction to remove the water used during catalyst loading. During the drying step, the reactor was kept under nitrogen flow (1000 cc/min & 40 psig) at room temperature for 2 hours. The reactor was then heated to 60° C. under nitrogen flow (1000 cc/min & 40 psig) at a rate of 1° C./min. The reactor was maintained at this temperature for 2 hours. Following this step, the reactor was heated to 120° C. under nitrogen flow (1000 cc/min & 40 psig) at a rate of 1.5° C./min and maintained at this temperature for 14 hours. The reactor was then pressurized to the desired reaction pressure (usually 350 psig). Syngas flow, with a 2:1 H₂/CO ratio, was fed to the reactor and the temperature was increased to the desired reaction temperature (usually 220° C.) at a rate of 1.5° C./min.

General Procedure for Material Balances

The reactors went through an unsteady state period of less than an hour during which time the CO conversion slowly increased. The hydrocarbon products collected from the wax and cold traps during the first four hours of reaction (including the unsteady-state period) were usually not included in a material balance period. The first material balance period started at about four hours subsequent to the start of the reaction.

A material balance period lasted for between 16 to 24 hours. During the material balance period, data was collected for feed syngas and exit uncondensed gas flow rates and compositions, weights and compositions of aqueous and organic phases collected in the wax and cold traps, and reaction conditions such as temperature and pressure. The information collected was then analyzed to get a total as well as individual carbon, hydrogen and oxygen material balances. Thus complete information was obtained regarding the type and quantities of reactor inputs (CO and H₂) as well as the type and quantities of reactor outputs (hydrocarbon products, water, oxygenates & unconverted reactants). From this information, properties such as CO Conversion (%), Selectivity/Alpha plot for all (C₁ to C₄₀) of the hydrocarbon products, C₅₊ Productivity (g/hr/kgcat), weight percent CH₄ in hydrocarbon products (%), etc., were obtained.

Usually, material balances were obtained for each catalyst under so-called standard reaction conditions: 220° C., 350 psig, syngas space velocity of 2 Nl/hr/gcat and H₂/CO ratio of 2. Reaction conditions were then varied, if necessary, to obtain different activity/selectivity at other than standard conditions.

General Regeneration Procedure

A regeneration procedure included at least one or both of the following two steps: 1.) Stripping of catalyst with hydrogen at reaction temperature & pressure for 16 hours; 2.) Reduction with hydrogen at 280 C and reaction pressure for 16 hours. This procedure was carried out in-situ.

Exemplary Catalysts

Catalyst 1

A Raney™ 2789 catalyst, was obtained from Grace Davison. The catalyst is a fixed bed unpromoted Raney cobalt catalyst having a particle size of 3-8 mesh (2.4 mm -6.7 mm). This catalyst is described in Grace Davison Raney Technical Manual, 4^{th} edition, pages 21-26, hereby incorporated herein by reference.

Catalyst 2

A promoted sponge Co catalyst was obtained from Grace Davison. The catalyst was made by incorporating the promoter by post-treatment of a commercial unpromoted sponge cobalt, in particular a Raney 2789 catalyst, as used for Catalyst 1, and obtainable from Grace Davison. Catalyst 2 had a nominal composition, by weight, of 0.1% Ru, 3.4% aluminum, with the balance cobalt.

Catalyst 3

Raney™ 2724 was obtained from Grace Davison. The catalyst is a slurry phase Raney™ cobalt catalyst promoted with Ni and Cr. This catalyst is described in Grace Davison Raney™ Technical Manual, 4^{th} edition, which is incorporated herein by reference.

Catalyst 4:

A Mo and Ru promoted sponge Co catalyst was made from a commercially obtained alloy having the nominal composition: 57.6% Aluminum, 35.4% Cobalt, 5.3% Molybdenum and 1.7% Ruthenium. The alloy was treated with sodium hydroxide to remove most of the aluminum. The resulting catalyst had a nominal composition by weight of 11% Mo, 3.5% Ru, 6% Al, with the balance being Co.

### EXAMPLES 1 and 2

Catalysts 1 and 2 were each tested in a fixed bed reactor, using the general fixed bed and general material balance procedures described above. For purposed of comparison, equal weights of catalysts were tested. Three different measures of the product distribution were determined, namely C₅₊ productivity, α, and C₁ wt %. Cₙ refers to hydrocarbons containing n carbon atoms. The C₅₊ productivity is given as grams-product/h/kilograms-catalyst. α was determined from the slope of a least squares fit to the data for log(Wₙ/n) vs. n, where Wₙ is the weight fraction of Cₙ product, and n was between 3 and 38. The C₁ wt % is also a measure of methane selectivity. It is defined as the weight percent of methane in total hydrocarbon products. Lower values are preferred for a process selective to C₅₊ hydrocarbons.

Results of testing catalysts 1 and 2 are shown in Tables 1 and 2, respectively. The catalysts were each tested under similar conditions of temperature and pressure. It can be seen that the value of cc for each catalyst remains over time at near 0.8. Catalyst 2 has a higher C₅₊ productivity than that of Catalyst 1. The C₁ wt % rises above 20% for Catalyst 1 and remains below 20% for Catalyst 2. The % CO conversion using Catalyst 2 is higher than when using Catalyst 1 during a similar period of time.

From the results shown in Tables 1 and 2, for Fischer-Tropsch synthesis process carried out under these or equivalent conditions in a fixed bed reactor or equivalent, Catalyst 2 containing ruthenium/cobalt is preferred to the unpromoted sponge cobalt catalyst, Catalyst 1. Further testing of a sponge cobalt catalyst containing 5% Ru/2.5% Ni/3% Al/89.5% Co, described in U.S. Provisional Patent Application No. 60/272,281, which is incorporated herein by reference showed higher methane production that Catalyst 2, as would be expected. As methane is undesirable, this catalyst is less preferred than Catalyst 1 or Catalyst 2.

Comparative testing of conventional catalysts showed that Catalyst 2 has comparable rates of % CO conversion. However, conventional catalysts are typically activated by a high-temperature activation that includes reduction in hydrogen prior to reaction. An advantage of Catalysts 1 and 2 is their comparable rate of conversion in the absence of activation prior to reaction.

It will be understood that C₂-C₄ selectivity is readily determined as the total selectivities, in wt %, must sum to 1. Further, the weight percent of different C₅₊ fractions than those tabulated may be determined from the data given, using the value of α given. For example, when the C₂₊ hydrocarbons approximately follow the Anderson-Shultz-Flory distribution, the weight fractions of individual hydrocarbons having n carbon atoms per molecule can be calculated from α and C₁ wt % (denoted C₁ in the following formula) using the following formula:

$${w}_{n} = ( 1 - \frac{{C}_{1}}{100} ) \frac{n {{α}^{n - 1} ( 1 - α )}^{2}}{1 - {( 1 - α )}^{2}} .$$

TABLE 1
Catalyst 1
Raney ™ 2789 Co Sponge

| Age | T | P | % CO | C₅₊ | | C₁ | C₅-C₁₂ | C₁₃-C₂₀ | C₂₁₊ |
|---|---|---|---|---|---|---|---|---|---|
| (hrs) | (° C.) | (psig) | conv. | (g/h/kg) | α | (wt %) | (wt %) | (wt %) | (wt %) |
| 27 | 219 | 358 | 45.7 | 107.0 | 0.81 | 14.8 | 52.4 | 17.5 | 3.6 |
| 59 | 220 | 355 | 42.5 | 85.5 | 0.79 | 25.9 | 36.8 | 12.1 | 3.0 |

TABLE 2
Catalyst 2
0.1% Ru/3.4% Al/96.5% Co Sponge

| Age | T | P | % CO | C₅₊ | | C₁ | C₅-C₁₂ | C₁₃-C₂₀ | C_{21 +} |
|---|---|---|---|---|---|---|---|---|---|
| (hrs) | (° C.) | (psig) | conv. | (g/h/kg) | α | (wt %) | (wt %) | (wt %) | (wt %) |
| 22 | 220 | 340 | 72.7 | 126.0 | .81 | 16.6 | 38.6 | 11.3 | 3.8 |
| 47 | 220 | 352 | 72.8 | 115.6 | .82 | 13.3 | 32.4 | 8.6 | 3.6 |
| 71 | 220 | 353 | 62.2 | 111.7 | .81 | 16.9 | 37.0 | 8.5 | 3.5 |
| 96 | 220 | 351 | 61.3 | 106.1 | .81 | 17.5 | 37.8 | 9.0 | 3.4 |
| 119 | 219 | 355 | 54.7 | 99.3 | .82 | 17.5 | 37.5 | 8.8 | 3.7 |
| 142 | 220 | 355 | 55.0 | 95.1 | .81 | 18.2 | 35.6 | 8.4 | 3.2 |
| 166 | 220 | 356 | 56.2 | 101.9 | .82 | 17.4 | 36.0 | 9.0 | 4.3 |

### EXAMPLE 3

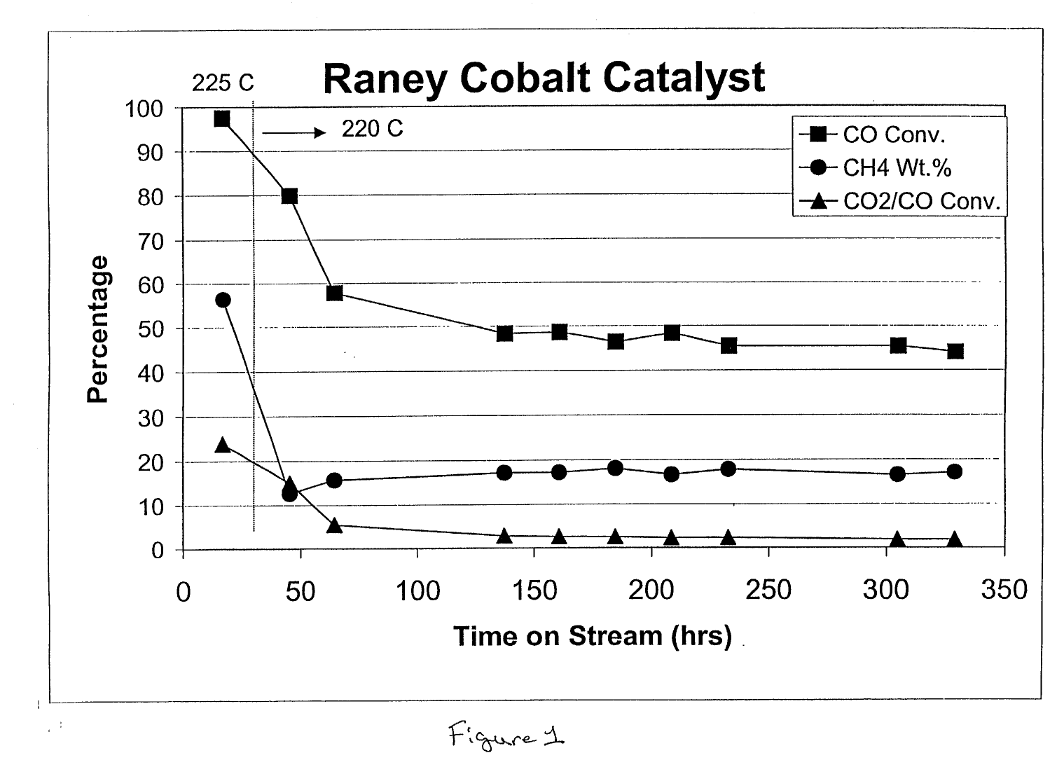

Catalyst 3 was tested in a continuously stirred tank reactor (CSTR) containing catalyst in a slurry with hydrocarbon wax, using the general slurry bed testing procedure and general material balance procedures described above. Results are shown in FIG. 1 and Table 3.

TABLE 3
Catalyst 3
Raney ™ 2724 (Cr/Ni/Co/Al) Sponge

| Age | T | P | % CO | C₅₊ | | C₁ |
|---|---|---|---|---|---|---|
| (hr) | (° C.) | (psig) | Conv. | (g/h/kg) | α | (Wt. %) |
| 16.75 | 225 | 350 | 97.52 | 39.94 | 0.69 | 56.39 |
| 45.50 | 220 | 350 | 79.98 | 157.85 | 0.72 | 12.63 |
| 64.50 | 220 | 350 | 57.79 | 103.38 | 0.72 | 15.65 |
| 137.50 | 220 | 350 | 48.33 | 85.61 | 0.72 | 17.17 |
| 160.50 | 220 | 350 | 48.63 | 93.89 | 0.72 | 17.21 |
| 184.50 | 220 | 350 | 46.40 | 88.97 | 0.72 | 18.10 |
| 208.50 | 220 | 350 | 48.33 | 98.27 | 0.72 | 16.63 |
| 232.75 | 220 | 350 | 45.43 | 86.92 | 0.72 | 17.84 |
| 304.75 | 220 | 350 | 45.39 | 92.12 | 0.72 | 16.56 |
| 328.75 | 220 | 350 | 44.07 | 91.58 | 0.72 | 17.17 |
| 357.75 | 220 | 350 | 43.10 | 92.18 | 0.72 | 17.27 |
| 376.50 | 220 | 350 | 43.28 | 91.25 | 0.72 | 17.31 |
| 497.25 | 220 | 350 | 53.87 | 114.85 | 0.72 | 12.41 |
| 520.75 | 220 | 350 | 49.63 | 103.70 | 0.72 | 14.05 |
| 545.00 | 220 | 350 | 49.14 | 107.69 | 0.72 | 13.88 |
| 569.00 | 220 | 350 | 48.45 | 109.92 | 0.72 | 13.80 |
| 640.75 | 220 | 350 | 47.15 | 101.86 | 0.72 | 14.11 |
| 664.75 | 220 | 350 | 43.74 | 90.94 | 0.72 | 15.93 |
| 688.75 | 220 | 350 | 43.78 | 92.74 | 0.72 | 15.88 |
| 712.50 | 220 | 350 | 44.93 | 105.55 | 0.72 | 15.06 |
| 736.50 | 220 | 350 | 41.71 | 80.36 | 0.72 | 16.72 |

### EXAMPLE 4

### 11% Mo/3.5% Ru/6% Al/79.5% Co Sponge

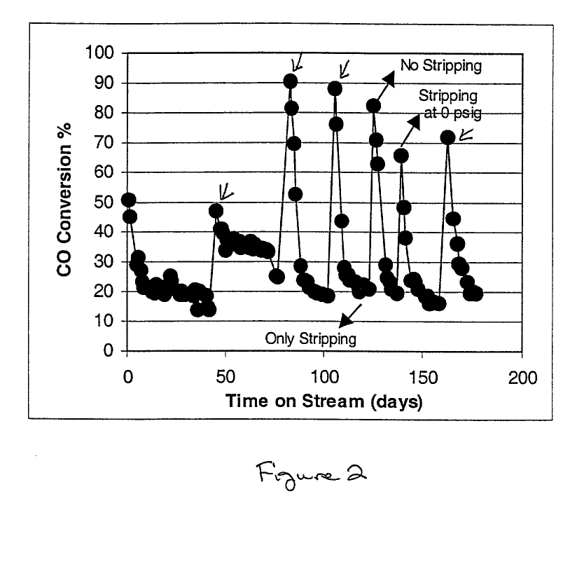

FIG. 2 shows the results of a regeneration procedure developed carried out over Catalyst 4 in situ in a slurry phase CSTR reactor as described. The regeneration procedure described above was cycled with reaction using the general slurry bed testing procedure described above. The reaction conditions were 220 C, 350 psig and a space velocity=2 NL/h/gcat.

As shown in FIG. 2, the two-step regeneration procedure, including stripping and reduction, works very well and returns the catalyst to its starting activity. The large increase in CO conversion after the second regeneration may be due to catalyst re-structuring. Further tests were carried out using each step of the regeneration procedure in the absence of the other. Only stripping (with hydrogen) at reaction temperature and pressure did not do any catalyst regeneration. Only step 2, reduction with hydrogen at 280 C & reaction pressure, did regenerate the catalyst. However, the catalyst activity was not fully regained.

A different regeneration was tried which involved only stripping but at atmospheric pressure and reaction temperature. The catalyst regained some of its activity with stripping at atmospheric pressure. The final regeneration was done following the two-step procedure, once again. The catalyst regained only part of its activity.

Though the regeneration procedure worked well for a number of times, it appears that the efficacy of the regeneration procedure decreases with the number of times it is done. The CO conversion immediately following regeneration showed a downward trend as shown in FIG. 2.

A comparison of stripping at atmospheric pressure & reaction temperature with the two-step procedure indicates that the two-step regeneration procedure was more effective. This can be quantified approximately by assuming that the gain (CO conversion) in catalyst activity after regeneration followed a linear decline with time on stream of the catalyst. Thus, following stripping at atmospheric pressure, the catalyst should have shown a CO conversion of 80%. However, the conversion exhibited was only 65%. Hence stripping at atmospheric pressure & reaction temperature led to incomplete regeneration.

While preferred embodiments of this invention have been shown and described, modifications thereof can be made by one skilled in the art without departing from the spirit or teaching of this invention. The embodiments described herein are exemplary only and are not limiting. Many variations and modifications of the method are possible and are within the scope of the invention. In particular, where steps of method are listed, they may be carried out in any order, unless specified otherwise. Accordingly, the scope of protection is not limited to the embodiments described herein, but is only limited by the claims that follow, the scope of which shall include all equivalents of the subject matter of the claims.

We claim:

1. A process for producing hydrocarbons, comprising contacting a feed stream comprising hydrogen and carbon monoxide with a catalyst in a reaction zone, wherein the catalyst comprises a sponge; wherein the catalyst comprises between about 85 and 99 wt % cobalt and between about 1 and about 15% stabilizing material; and wherein the process is adapted to produce hydrocarbons suitable for the production of diesel fuel.

2. The process according to claim 1 wherein the hydrocarbons are described by an α value of at least about 0.72.

3. The process according to claim 1 wherein the selectivity to methane is up to about 20 wt %.

4. The process according to claim 1 wherein the selectivity to wax is up to about 5 wt %.

5. The process according to claim 1 wherein the selectivity to wax is greater than about 5 wt %.

6. The process according to claim 1 wherein the reaction zone comprises a slurry bubble column.

7. The process according to claim 1, wherein the reaction zone comprises a fixed bed reactor.

8. The process according to claim 1 wherein the stabilizing material selected from the group consisting of aluminum, silicon, titanium, zirconium, and combinations thereof.

9. The process according to claim 8 wherein stabilizing material comprises aluminum.

10. The process according to claim 1 wherein the catalyst further comprises from about 0.05 to about 6 wt. % of a promoter.

11. The process according to claim 10 wherein the promoter is selected from the group consisting of Groups 2-15 of the Periodic Table.

12. The process according to claim 11 wherein the promoter is selected from the group consisting of chromium, iron, molybdenum, nickel, palladium, platinum, rhenium, rhodium, ruthenium, and combinations thereof.

13. The process according to claim 12 wherein the promoter comprises ruthenium.

14. The process according to claim 10 wherein the catalyst comprises between about 0.05 and about 0.3 wt. percent promoter.

15. A process for producing hydrocarbons, comprising contacting a feed stream comprising hydrogen and carbon monoxide with a catalyst in a reaction zone, wherein the catalyst comprises a sponge; wherein the catalyst comprises between about 85 and about 99 wt. % cobalt and between about 1 and about 15 wt. % stabilizing material selected from the group consisting of aluminum, silicon, zirconium, titanium, and combinations thereof, and wherein the hydrocarbons are described by an α value of at least about 0.72, and wherein the selectivity to methane is up to about 20 weight percent.

16. The process according to claim 15 wherein the selectivity to wax is up to about 5 wt %.

17. The process according to claim 15 wherein the selectivity to wax is greater than about 5 wt %.

18. The process according to claim 15 wherein the reaction zone comprises at least one slurry bubble column.

19. The process according to claim 15 wherein the reaction zone comprises at least one fixed bed reactor.

20. The process according to claim 15 wherein catalyst further comprises between about 0.04 and about 6 wt % of promoter selected from the group consisting of Groups 2-15 of the Periodic Table.

21. A process for producing hydrocarbons, comprising:
a) contacting a feed stream comprising carbon monoxide and hydrogen with a sponge catalyst comprising cobalt in a reaction zone under reaction conditions effective to produce an effluent stream comprising C₅-C₂₀ hydrocarbons;
b) regenerating the catalyst; and
c) cycling between steps a and b.

22. The process according to claim 21 wherein step (b) comprises:
1) stripping the catalyst with hydrogen at reaction conditions;
2) reducing the catalyst with hydrogen at a temperature elevated above the reaction temperature and at reaction pressure.

23. The process according to claim 21 wherein step (b) is carried out in situ.

24. The process according to claim 21 wherein step (b) is carried out ex situ.

25. The process according to claim 24 wherein step (b) comprises:
1) withdrawing at least a portion of the catalyst from the reaction zone; and
2) passing the portion to an ex situ regeneration zone.

26. The process according to claim 21 wherein the hydrocarbons are described by an a value of at least about 0.72.

27. The process according to claim 21 wherein the selectivity to methane is up to about 20 wt %.

28. The process according to claim 21 wherein the reaction zone comprises at least one slurry bubble column.

29. The process according to claim 21 wherein the reaction zone comprises at least one fixed bed reactor.
